(19) United States
(12) Plant Patent Application Publication
(10) Pub. No.: US 2014/0143919 P1
(43) Pub. Date: May 22, 2014

(54) Walnut tree named 'Solano'

(50) ;

(71) Applicant: THE REGENTS OF THE UNIVERSITY OF CALIFORNIA, Oakland, CA (US)

(72) Inventors: Gale McGranahan, Davis, CA (US); Charles Leslie, Davis, CA (US)

(73) Assignee: The Regents of the University of California, Oakland, CA (US)

(21) Appl. No.: 13/694,337

(22) Filed: Nov. 20, 2012

(51) Int. Cl. A01H 5/00

(52) U.S. Cl. PLT/154

(57) ABSTRACT

A new and distinct cultivar of walnut tree denominated ‘Solano’ is described. This new cultivar, ‘Solano’, comes into bearing young, with an excellent yield. ‘Solano’ forms jumbo-sized walnuts that possess light-colored kernels with little size variation in a given harvest. ‘Solano’ bears fruit terminally and laterally and yields a crop that can be harvested approximately 2 weeks before ‘Chandler’ (U.S. Plant Pat. No. 4,388). ‘Solano’ is also protandrous, bearing male flowers before female flowers.

### LATIN NAME

Botanical/commercial classification: (Juglans regia), new English walnut tree cultivar.

### VARIETAL DENOMINATION

The varietal denomination of the claimed walnut is ‘Solano’.

### BACKGROUND OF INVENTION

The present invention relates to a new and distinct cultivar of walnut tree (Juglans regia) that has been denominated as ‘Solano’ and more particularly to a walnut tree that has a harvest date approximately 2 weeks earlier than the walnut tree cultivar ‘Chandler’ (U.S. Plant Pat. No. 4,388), and that further produces a walnut that is jumbo in size with light colored kernels.

It has long been recognized as desirable to provide walnut trees bearing large crops that are ripe for commercial harvesting and shipment early in the harvest season. The tree of the present cultivar, ‘Solano’, produces a nut that has a quality similar to the highest quality cultivar ‘Chandler’ (U.S. Plant Pat. No. 4,388). However, the new cultivar is ready for harvest approximately 2 weeks before ‘Chandler’, which is about the same time as the reference cultivar ‘Vina’ (old cultivar; not patented).

The new Juglans regia walnut tree of the present invention was created at Davis, Calif. in Year 1 by a controlled pollination between the cultivar ‘Chico’ and ‘UC67-13’ (neither patented). The pedigree is illustrated (FIG. 1).

Seeds from the cross were planted and the resulting 29 trees were carefully observed along with other trees in the walnut breeding program. When the trees began to bear nuts, data were collected annually on leafing date, first, peak and last female flower bloom, first, peak and last male bloom, blight severity, and yield (Table 1). Nuts were sampled, cracked, and data was collected on shell appearance, shell thickness, shell integrity, shell strength, nut weight, kernel weight, percent kernel, ease of kernel removal, kernel color, and percent kernel shrivel (Table 1). A single tree was selected from among progeny of this controlled cross based on its superior attributes. This selection was originally designated ‘UC95-11-16’ and is now designated as the ‘Solano’ cultivar after the county in California. Compared to ‘Solano’, the parent ‘UC67-13’ has larger nuts, an earlier harvesting date, and is susceptible to pistillate flower abscission. The parent ‘Chico’ is protogynous and has smaller nuts than ‘Solano’ with a more difficult to extract kernel (Table 1).

The new ‘Solano’ cultivar of the present invention has been asexually reproduced by grafting at Davis, Calif. and in various field trials on ‘Paradox’ rootstock. The distinctive characteristics of the new cultivar have been found to be stable and are transmitted to the new trees when asexually propagated.

### SUMMARY OF THE INVENTION

It was found that the walnut cultivar ‘Solano’ of the present invention exhibits the following combination of characteristics:
a) comes into bearing young, with an excellent yield;
b) forms jumbo-sized walnuts that possess light-colored kernels with little size variation in a given harvest;
c) bears fruit terminally and laterally;
d) yields a crop that can be harvested approximately 2 weeks before ‘Chandler’ (U.S. Plant Pat. No. 4,388); and
e) is protandrous, bearing male flowers before female flowers.

### BRIEF DESCRIPTION OF THE TABLE

Table 1 shows tree and nut evaluations for both parents and the most common walnut cultivar ‘Chandler’.

### BRIEF DESCRIPTION OF THE DRAWINGS

FIG. 1 shows pedigree of the ‘Solano’ walnut tree.

FIG. 2 shows a ‘Solano’ walnut tree at 8 years old.

FIG. 3 shows bark of a ‘Solano’ walnut tree.

FIG. 4 shows an adaxial view of a typical leaf of a ‘Solano’ walnut tree.

FIG. 5 shows an abaxial view of a typical leaf of a ‘Solano’ walnut tree.

FIG. 6 shows female flowers of a ‘Solano’ walnut tree.

FIG. 7 shows catkins (male flowers) of a ‘Solano’ walnut tree.

FIG. 8 shows hulls from a ‘Solano’ walnut tree before opening and exposing the nuts.

FIG. 9 shows nuts from a ‘Solano’ walnut tree.

FIG. 10 shows kernels from a ‘Solano’ walnut tree.

### BOTANICAL DESCRIPTION OF THE PLANT

The following description is based on an ungrafted walnut tree on its own roots and trees subsequently grafted on ‘Paradox’ rootstock in selection blocks at Davis, Calif., Chico, Calif., and Parlier, Calif.; as well as in grower trials near Madison, Calif., near Durham, Calif., and near Wheatland, Calif. The first data were collected on the own rooted tree from Year 4 at age 3 years, to Year 11, age 10. Data from Year 9 to Year 15 were collected from grafted trees.

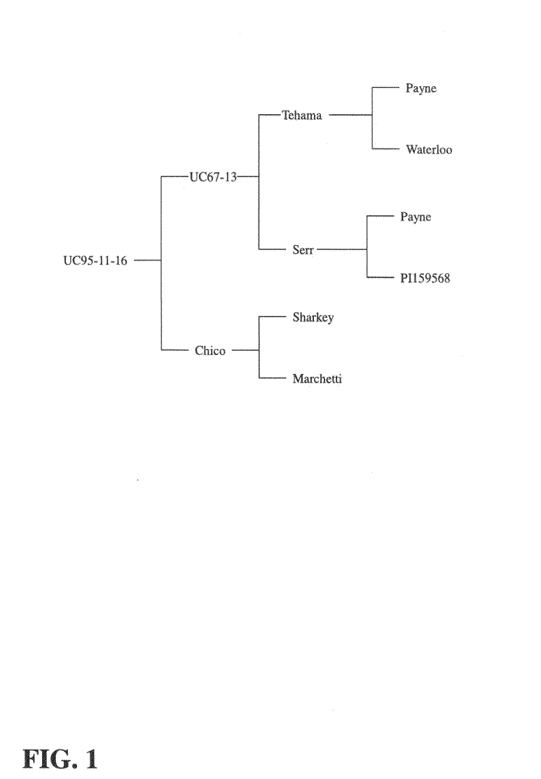

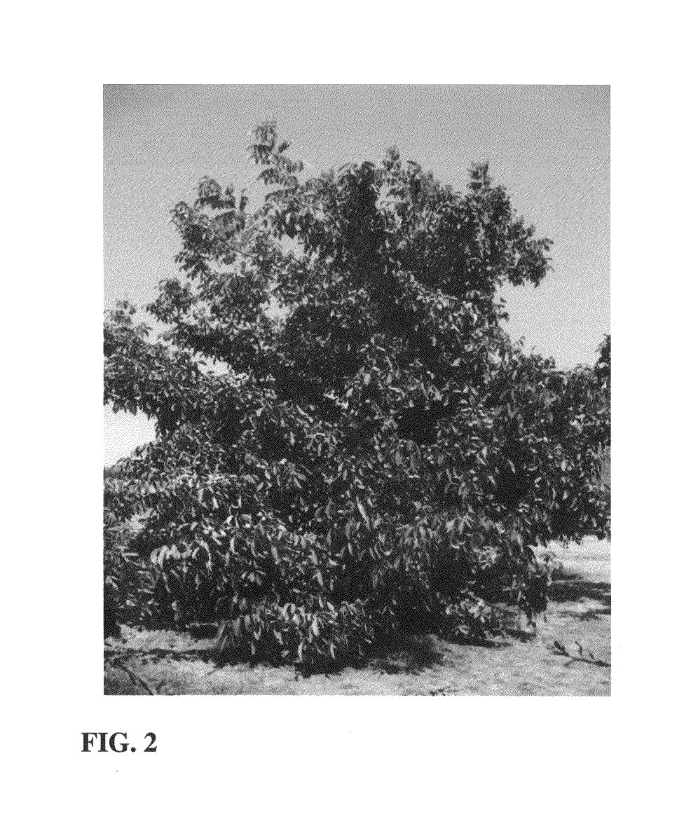

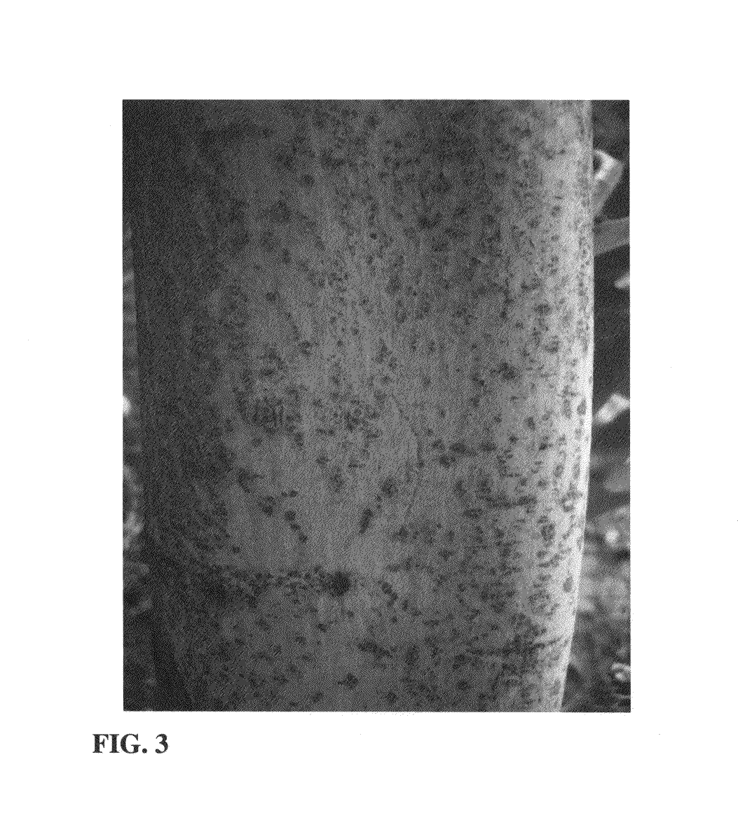

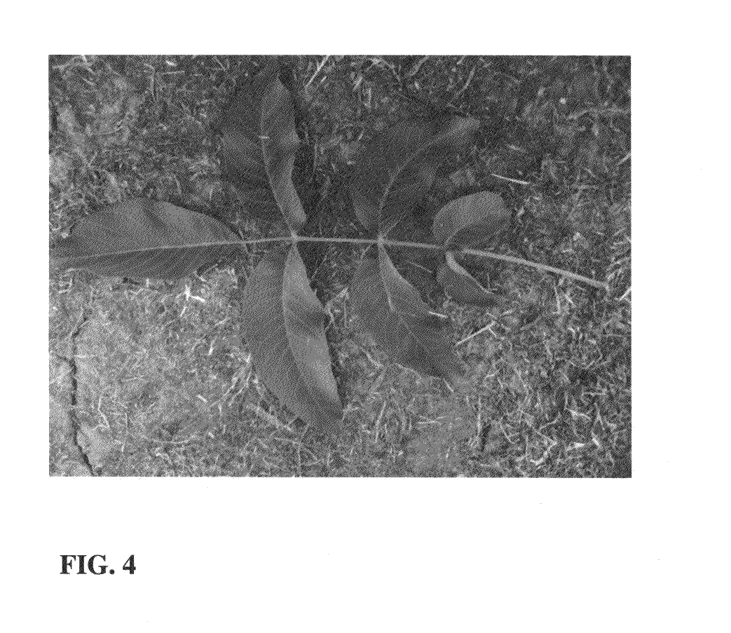

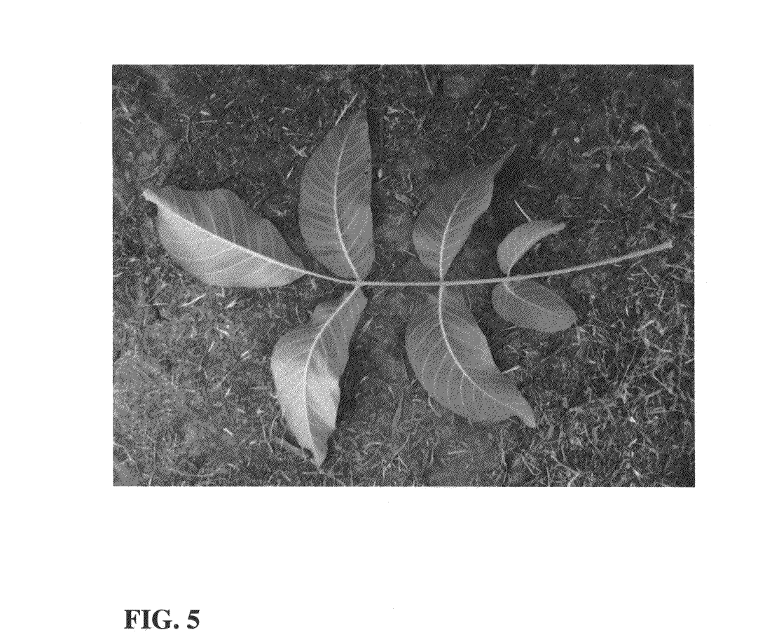

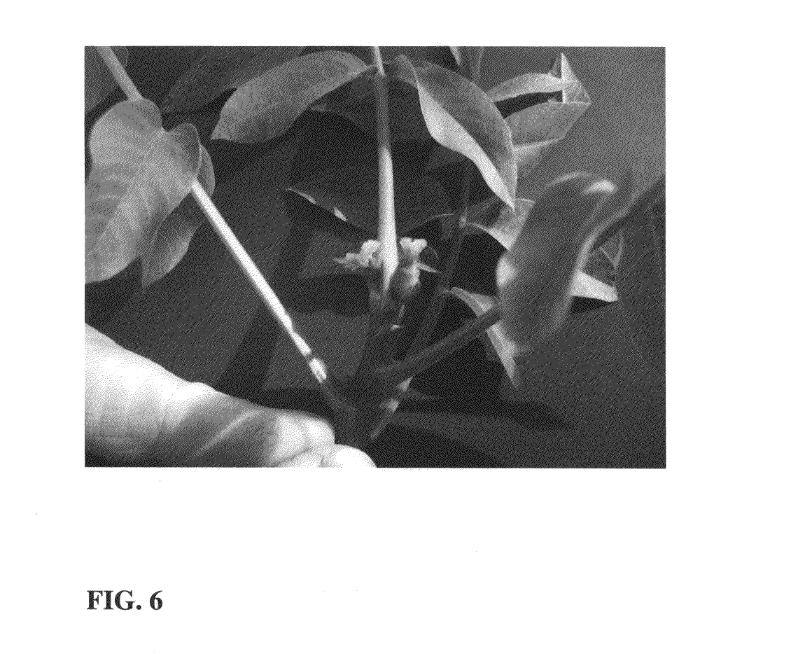

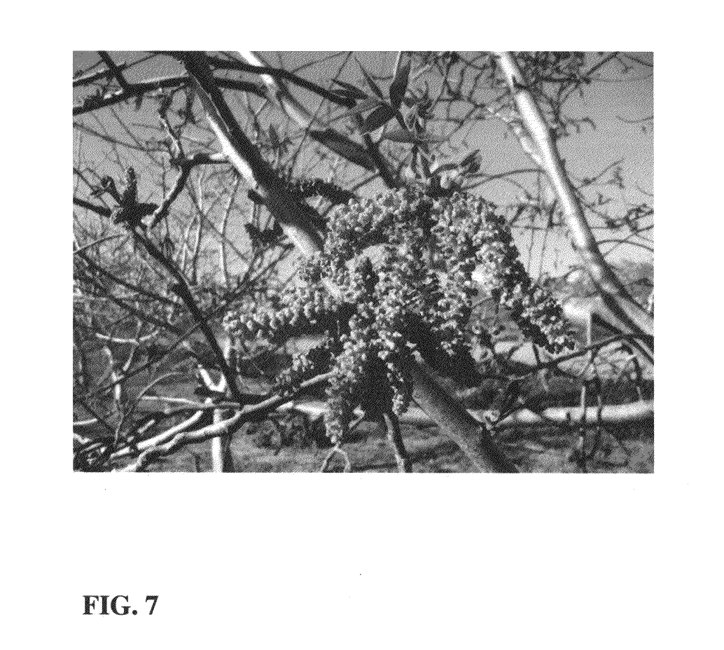

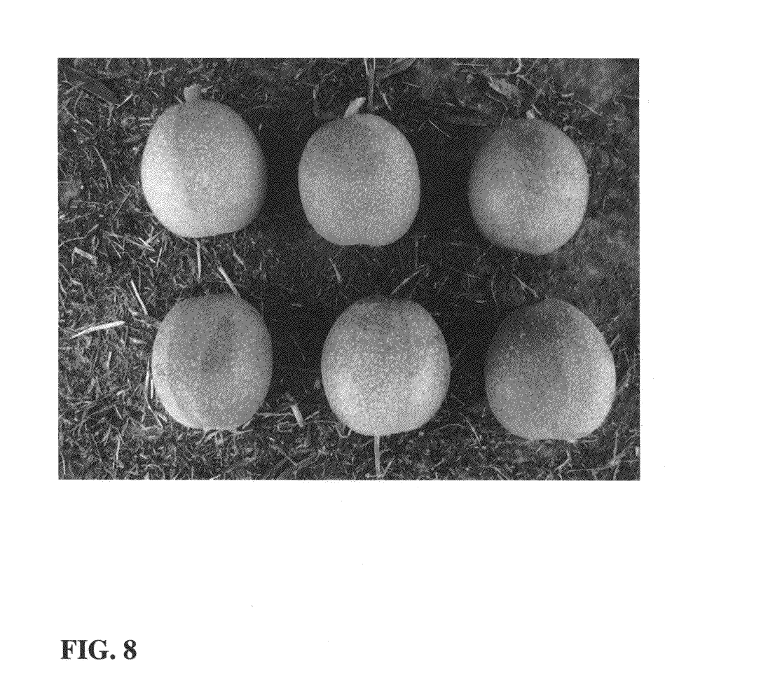

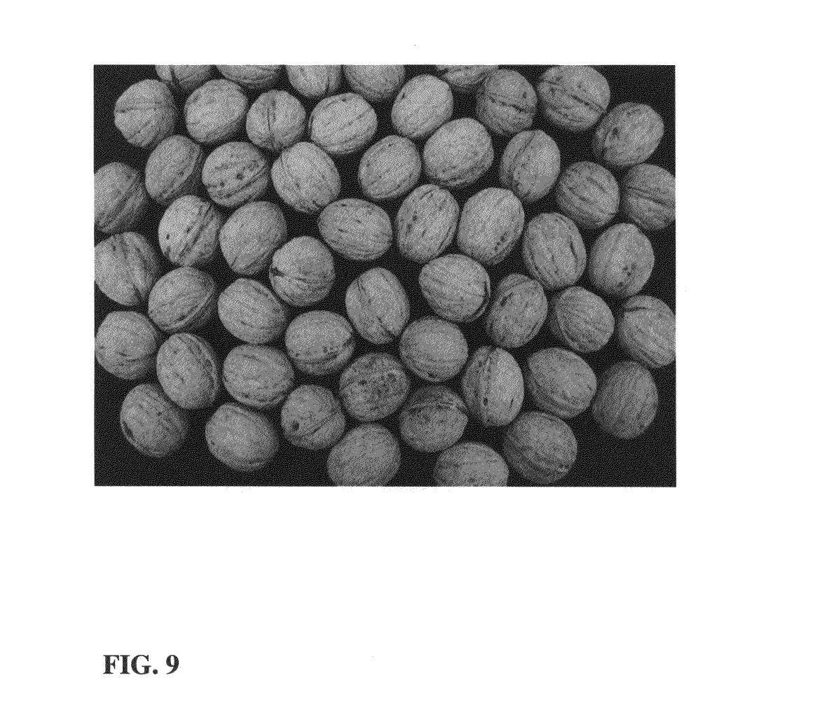

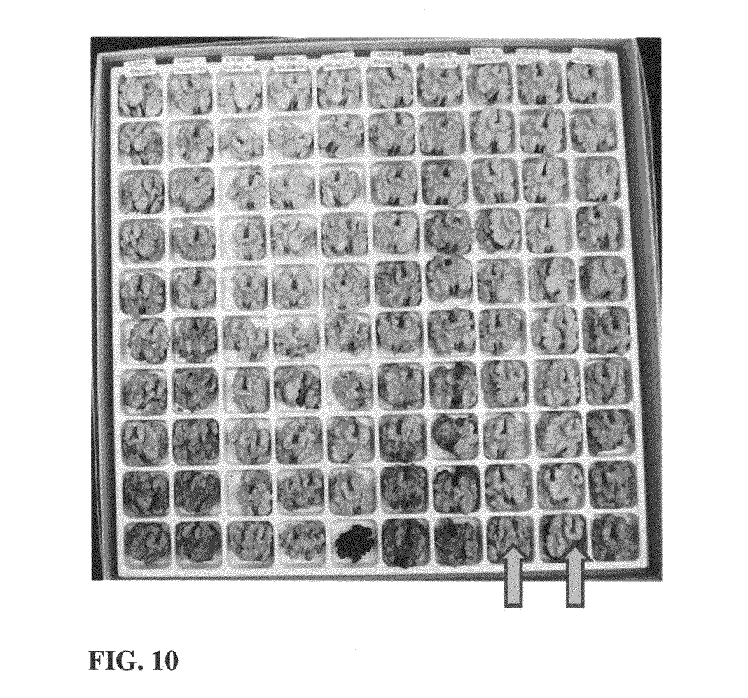

The Munsell Book of Color was used in the identification of color. Also, common color terms are to be accorded their ordinary dictionary significance.
Botanical classification: Juglans regia.
   Female parent.—‘UC67-13’.
   Male parent.—‘Chico’. The pedigree is shown in FIG. 1.
Plant: The growth habit of the plant is illustrated in FIG. 2. This 8 year old tree is approximately 7.2 meters in height with a canopy diameter of 8.1 meters. The trunk diameter at 0.5 meters above the ground is approximately 24 cm. The silvery grey bark is typical of Juglans regia. The young bark is brown (5Y 5/2) and the older bark is grey (5Y 7.5/2) with raised lighter lenticels (2.5Y 8/2) (FIG. 3). Lenticels are round to oval in shape, 1-10 mm×1-3 mm in size. ‘Solano’ has vigor similar to ‘Chandler’.
Foliage: The dark green foliage is illustrated in FIG. 4 and FIG. 5, and is typical of Juglans regia. Leafing out date between Year 4 and Year 15 has occurred on March 25^{th} on the average. For comparative purposes the ‘Chandler’ cultivar leaf-out is April 4^{th}. The typical leaf coloration is green (adaxial 5GY 3/4, abaxial 5GY 5/4). The leaves are pinnately compound with 7-9 leaflets. The full leaf length is approximately 38 cm and the width is 30 cm. Leaflets are broadly elliptical and entire. The terminal leaflet averages 17 cm in length and 9 cm in width. The middle leaflets average 15 cm in length and 6 cm in width, and the proximal leaflets average 6 cm in length and 3.5 cm in width. The rachis averages 22 cm in length and 2-4 mm in diameter. Petiole length is 7 cm and is 2.5GY 7/6 in color.
Inflorescence: The tree is precocious with excellent yield being noted at age 3 years. Male flowers (catkins) were first present at age 4 years. This delay in male maturity is typical of Juglans regia. From Year 4 to Year 15, average first female bloom occurred on April 9^{th}, peak bloom on April 13^{th} and last bloom on April 19^{th}. From Year 5 to Year 15, average male flowering (pollen shedding) began March 28^{th}, peaked on April 2^{nd}, and terminated on April 11^{th}. Pollen shedding in this protandrous cultivar does not completely cover pistillate bloom, suggesting that a pollenizer would be needed for maximum yield in isolated areas. ‘Chandler’ (U.S. Plant Pat. No. 4,388), ‘Howard’ (U.S. Plant Pat. No. 4,405), and ‘Tulare’ (U.S. Plant Pat. No. 8,268), would all be satisfactory pollenizers. The female flowers are typical of Juglans regia (FIG. 6), with 2-3 flowers per inflorescence borne on 1 cm spike at both terminal and lateral positions on current season's growth. Approximately 100% of the lateral buds contain inflorescences making yields much greater than trees that only bear flowers terminally. A typical female flower is approximately 5 to 7 mm at anthesis and floral organs are typical of Juglans regia. The flowers appear vase-shaped when the 2 plumose stigmatic arms are curved outward. There are no petals. The female flowers are green in color (5GY 7/8). The flower fragrance is typical of Juglans regia and is not noticeably different than the foliage fragrance. The male flowers (FIG. 7) are borne on catkins, between 7.5 and 14 cm in length and 1.5 cm in diameter, and are green in color (5GY 7/8).
Walnuts: The new cultivar ‘Solano’ commonly harvests about 2 weeks before ‘Chandler’, about one week after ‘Payne’, and is similar in timing to ‘Vina’. The new cultivar has excellent yields of mostly jumbo-sized walnuts. The hull is globose, 5.5 cm×5.0 cm, 6 mm thick, and 2.5GY 6/6 in color with numerous lighter speckles (FIG. 8). The nut is broadly elliptical, has a slightly rough but uniform and attractive appearance, is tan in color (7.5YR 6/4), and measures approximately 42 mm in length and 36 mm in width (FIG. 9). The shell is 1.3 mm thick, relatively strong and well-sealed, suitable for in-shell marketing, and the kernel is easy to remove. The kernel weight averages 8.0 g and makes up 55% of the total nut weight of 14.6 g. Kernel color is considered excellent (FIG. 10), and scores mostly in the light to extra light categories of the USDA Standards for Grades of Shelled Walnuts as determined by using the standard Walnut Color Chart for kernels published by the Dried Fruit Association of California. In addition, kernels of ‘Solano’ have averaged 54.8 on the Relative Light Index used by Diamond Foods of Stockton, Calif. Walnuts of the new cultivar ‘Solano’ are typical of commercial walnuts in terms of flavor and firmness, the latter varying according to the percent moisture after drying.
Disease susceptibility: The response of the new cultivar ‘Solano’ to Xanthomonas campestris pv. juglandis and walnut husk fly (Rhagoletis completa) appears to be typical of mid-season cultivars.
Usage: The new cultivar ‘Solano’ of the present invention provides a relatively early to mid-season walnut with high quality light-colored kernels that can be used cracked or in-shell.

TABLE 1
Performance of ‘Solano’ compared to ‘UC67-13’,
‘Chico’, ‘Chandler’, and ‘Vina’.

| | | Variety | | | | | |
|---|---|---|---|---|---|---|---|
| Trait¹ | | Solano² | | | UC67-13³ | | |
| Leafing | | 3/25 | (1.83) | 22⁴ | 3/24 | (1.08) | 19 |
| date | | | | | | | |
| Pollen | First | 3/28 | (2.03) | 13 | 3/31 | (1.74) | 17 |
| shed | Peak | 4/2 | (1.98) | 13 | 4/6 | (1.52) | 17 |
| date | Last | 4/11 | (1.82) | 13 | 4/19 | (1.33) | 16 |
| Female | First | 4/9 | (1.58) | 18 | 4/6 | (1.31) | 17 |
| bloom | Peak | 4/13 | (1.50) | 18 | 4/12 | (1.15) | 16 |
| date | Last | 4/19 | (1.67) | 18 | 4/19 | (1.14) | 16 |
| Harvest | | 9/24 | (1.20) | 36 | 9/21 | (1.64) | 17 |
| date | | | | | | | |
| Days | | 7 | (1.05) | 19 | 6 | (1.43) | 11 |
| after | | | | | | | |
| Payne | | | | | | | |
| leafing | | | | | | | |
| Days | | −9 | (0.91) | 19 | −12 | (1.19) | 11 |
| after | | | | | | | |
| Chandler | | | | | | | |
| leafing | | | | | | | |
| Catkin | | 5.2 | (0.61) | 14 | 6.3 | (0.49) | 18 |
| abun- | | | | | | | |
| dance | | | | | | | |
| Female | | 6.3 | (0.22) | 16 | 5.8 | (0.35) | 11 |
| abun- | | | | | | | |
| dance | | | | | | | |
| Flowers/ | | 2.0 | (0) | 17 | 2.0 | (0) | 16 |
| inflores- | | | | | | | |
| cence | | | | | | | |
| Percent | | 95.3 | (2.29) | 17 | 96.6 | (1.18) | 16 |
| lateral | | | | | | | |
| bud fruit- | | | | | | | |
| fulness | | | | | | | |
| Yield | | 6.4 | (0.18) | 32 | 5.6 | (0.31) | 17 |
| Shell | Texture | 4.9 | (0.07) | 35 | 5.0 | (0.67) | 10 |
| | Color | 4.7 | (0.10) | 35 | 4.8 | (0.42) | 10 |
| | Seal | 5.1 | (0.06) | 35 | 5.0 | (0) | 11 |
| | Strength | 5.0 | (0.06) | 35 | 5.1 | (0.30) | 11 |
| | Integrity | 7.0 | (0) | 35 | 7.0 | (0) | 5 |
| | Thick- | 1.3 | (0.02) | 35 | 1.5 | (0.13) | 11 |
| | ness | | | | | | |
| | Packing | 5.0 | (0) | 35 | 5.1 | (0.57) | 10 |
| | tissue | | | | | | |
| Kernel | Inshell | 14.6 | (0.24) | 35 | 15.3 | (1.78) | 11 |
| | weight | | | | | | |
| | Kernel | 8.0 | (0.16) | 35 | 8.5 | (1.55) | 11 |
| | weight | | | | | | |
| | Percent | 54.6 | (0.45) | 35 | 55.4 | (2.16) | 11 |
| | kernel | | | | | | |
| | Fill | 5.4 | (0.09) | 35 | 4.7 | (1.27) | 11 |
| | Plump- | 5.4 | (0.08) | 35 | 4.7 | (1.10) | 11 |
| | ness | | | | | | |
| | Ease of | 4.7 | (0.07) | 35 | 4.3 | (0.48) | 10 |
| | removal | | | | | | |
| | Percent | 0.6 | (0.40) | 35 | 2.7 | (6.47) | 4 |
| | blank | | | | | | |
| | Percent | 33.8 | (5.48) | 35 | 18.8 | (35.7) | 11 |
| | extra | | | | | | |
| | light | | | | | | |
| | Percent | 57.4 | (4.92) | 35 | 72.6 | (33.7) | 11 |
| | light | | | | | | |
| | Percent | 9.8 | (2.34) | 35 | 6.7 | (4.05) | 11 |
| | light | | | | | | |
| | amber | | | | | | |
| | Percent | 0 | 0 | 35 | 1.8 | (6.03) | 11 |
| | amber | | | | | | |
| | Percent | 11.8 | (3.2) | 35 | 1.1 | (3.51) | 10 |
| | tip | | | | | | |
| | shrivel | | | | | | |
| | Percent | 16.6 | (3.12) | 35 | 44.4 | (22.4) | 11 |
| | veins | | | | | | |
| | | Variety | | | | | |
| Trait¹ | | Chico³ | | | Chandler² | | |
| Leafing | | 3/22 | (1.13) | 27 | 4/4 | (0.72) | 80 |
| date | | | | | | | |
| Pollen | First | 4/7 | (1.30) | 23 | 4/6 | (0.83) | 75 |
| shed | Peak | 4/13 | (1.16) | 24 | 4/14 | (0.72) | 74 |
| date | Last | 4/20 | (1.01) | 23 | 4/21 | (0.81) | 73 |
| Female | First | 3/25 | (0.92) | 25 | 4/17 | (0.72) | 77 |
| bloom | Peak | 4/1 | (1.02) | 25 | 4/23 | (0.68) | 77 |
| date | Last | 4/9 | (1.15) | 25 | 4/30 | (0.71) | 76 |
| Harvest | | 9/18 | (1.76) | 24 | 10/8 | (0.72) | 91 |
| date | | | | | | | |
| Days | | 3 | (0.45) | 18 | 17 | (0.60) | 64 |
| after | | | | | | | |
| Payne | | | | | | | |
| leafing | | | | | | | |
| Days | | −15 | (0.97) | 18 | 0 | (0.34) | 64 |
| after | | | | | | | |
| Chandler | | | | | | | |
| leafing | | | | | | | |
| Catkin | | 5.3 | (0.28) | 23 | 4.9 | (0.20) | 75 |
| abun- | | | | | | | |
| dance | | | | | | | |
| Female | | 6.1 | (0.26) | 22 | 6.1 | (0.13) | 67 |
| abun- | | | | | | | |
| dance | | | | | | | |
| Flowers/ | | 2.0 | (0) | 25 | 2.0 | (0.01) | 70 |
| inflores- | | | | | | | |
| cence | | | | | | | |
| Percent | | 99.9 | (0.08) | 25 | 95.7 | (0.75) | 68 |
| lateral | | | | | | | |
| bud fruit- | | | | | | | |
| fulness | | | | | | | |
| Yield | | 6.3 | (0.26) | 27 | 5.7 | (0.12) | 92 |
| Shell | Texture | 4.2 | (0.15) | 17 | 4.7 | (0.05) | 87 |
| | Color | 4.6 | (0.15) | 17 | 4.3 | (0.06) | 87 |
| | Seal | 5.4 | (0.15) | 18 | 4.9 | (0.03) | 88 |
| | Strength | 5.7 | (0.11) | 18 | 4.5 | (0.07) | 88 |
| | Integrity | 7.1 | (0.10) | 14 | 7 .0 | (0) | 82 |
| | Thick- | 1.5 | (0.03) | 18 | 1.3 | (0.02) | 88 |
| | ness | | | | | | |
| | Packing | 5.6 | (0.12) | 17 | 4.7 | (0.05) | 86 |
| | tissue | | | | | | |
| Kernel | Inshell | 11.4 | (0.29) | 18 | 13.4 | (0.19) | 88 |
| | weight | | | | | | |
| | Kernel | 5.3 | (0.12) | 18 | 6.6 | (0.10) | 88 |
| | weight | | | | | | |
| | Percent | 47.1 | (0.57) | 18 | 49.5 | (0.27) | 88 |
| | kernel | | | | | | |
| | Fill | 5.7 | (0.47) | 13 | 4.5 | (0.06) | 88 |
| | Plump- | 4.5 | (0.20) | 18 | 4.3 | (0.06) | 87 |
| | ness | | | | | | |
| | Ease of | 6.5 | (0.17 | 17 | 3.9 | (0.05) | 87 |
| | removal | | | | | | |
| | Percent | 2.4 | (1.06) | 17 | 1.3 | (0.36) | 87 |
| | blank | | | | | | |
| | Percent | 9.4 | (5.60) | 17 | 50.8 | (4.22) | 86 |
| | extra | | | | | | |
| | light | | | | | | |
| | Percent | 76.0 | (7.41) | 17 | 44.2 | (3.82) | 86 |
| | light | | | | | | |
| | Percent | 12.0 | (4.81) | 17 | 4.6 | (1.32) | 86 |
| | light | | | | | | |
| | amber | | | | | | |
| | Percent | 2.6 | (2.03) | 17 | 0.4 | (0.21) | 86 |
| | amber | | | | | | |
| | Percent | 0 | (0) | 17 | 30.5 | (2.60) | 86 |
| | tip | | | | | | |
| | shrivel | | | | | | |
| | Percent | 19.2 | (5.48) | 17 | 18.3 | (2.78) | 85 |
| | veins | | | | | | |

| | |
|---|---|
| | Variety |
| Trait¹ | Vina³ |

| | | | | |
|---|---|---|---|---|
| Leafing | | 3/26 | (0.89) | 41 |
| date | | | | |
| Pollen | First | 3/28 | (0.99) | 39 |
| shed | Peak | 4/5 | (1.01) | 39 |
| date | Last | 4/14 | (1.04) | 39 |
| Female | First | 4/8 | (1.05) | 40 |
| bloom | Peak | 4/13 | (0.87) | 40 |
| date | Last | 4/21 | (0.93) | 40 |
| Harvest | | 9/23 | (1.06) | 49 |
| date | | | | |
| Days | | 8 | (0.47) | 39 |
| after | | | | |
| Payne | | | | |
| leafing | | | | |
| Days | | −9 | (0.62) | 39 |
| after | | | | |
| Chandler | | | | |
| leafing | | | | |
| Catkin | | 5.7 | (0.21) | 39 |
| abun- | | | | |
| dance | | | | |
| Female | | 6.6 | (0.16) | 35 |
| abun- | | | | |
| dance | | | | |
| Flowers/ | | 2.0 | (0) | 37 |
| inflores- | | | | |
| cence | | | | |
| Percent | | 93.4 | (1.92) | 34 |
| lateral | | | | |
| bud fruit- | | | | |
| fulness | | | | |
| Yield | | 6.5 | (0.14) | 51 |
| Shell | Texture | 5.0 | (0) | 49 |
| | Color | 5.2 | (0.07) | 49 |
| | Seal | 5.2 | (0.05) | 50 |
| | Strength | 5.1 | (0.04) | 50 |
| | Integrity | 7.0 | (0) | 46 |
| | Thick- | 1.4 | (0.02) | 50 |
| | ness | | | |
| | Packing | 5 | (0.03) | 49 |
| | tissue | | | |
| Kernel | Inshell | 12.8 | (0.24) | 50 |
| | weight | | | |
| | Kernel | 3.3 | (0.13) | 50 |
| | weight | | | |
| | Percent | 49.3 | (0.30) | 50 |
| | kernel | | | |
| | Fill | 5.1 | (0.08) | 50 |
| | Plump- | 4.8 | (0.08) | 50 |
| | ness | | | |
| | Ease of | 4.9 | (0.11) | 50 |
| | removal | | | |
| | Percent | 2.0 | (0.58) | 49 |
| | blank | | | |
| | Percent | 4.1 | (2.90) | 49 |
| | extra | | | |
| | light | | | |
| | Percent | 48.1 | (4.99) | 49 |
| | light | | | |
| | Percent | 45.1 | (4.86) | 49 |
| | light | | | |
| | amber | | | |
| | Percent | 2.5 | (1.33) | 49 |
| | amber | | | |
| | Percent | 2.3 | (0.91) | 49 |
| | tip | | | |
| | shrivel | | | |
| | Percent | 30.4 | (5.04) | 49 |
| | veins | | | |

¹See attached key for description of scores;
²Data Year 4 to Year 15;
³Data Year Minus 6 to Year 15;
⁴Data mean, (Std. Error), n

In Table 1 the data for ‘Solano’ was collected over 11 years, from Year 4 to Year 15. The data for ‘UC67-13’, ‘Chico’, ‘Chandler’, and ‘Vina’ was collected over the course of 21 years, from Year Minus 6 to Year 15. In Table 1, in the row “Days after Chandler leafing” negative numbers refer to days before ‘Chandler’ leafing. Additionally, in footnote 4 of Table 1, “n” refers to the number of observations. Accordingly, the data in Table 1 are listed as mean values of the number of observations, including the standard error (Std. Error) in parentheses.

KEY FOR TABLE 1

Tree Evaluation

Catkin abundance Male flower abundance: 3 low; 5 intermediate; 7 high

Female abundance Female flower abundance: 3 low; 5 intermediate; 7 high

Lateral fruitfulness % Percent of lateral buds with female flowers

Yield Yield: 3 low; 5 intermediate; 7 high

Nut and Kernel Traits

Texture Shell texture: 3, smooth; 5 medium; 7 rough

Color Shell color: 3 light; 5 medium; 7 dark

Seal Shell seal: 3 weak; 5 intermediate; 7 strong

Strength Shell strength: 3 weak; 5 intermediate; 7 strong

Integrity Shell integrity: 3 substantial areas of shell missing;
5 small areas of shell missing; 6 hole in stem end; 7 complete shell

Thickness Shell thickness at mid-cheek in mm

Packing tissue Inner lining: 3 thin; 5 medium; 7 thick

Inshell weight g

Kernel weight g

Kernel % Kernel wt/inshell wt×100

Fill Kernel fill: 3 poor; 5 moderate; 7 well

Plumpness Kernel plumpness: 3 thin; 5 moderate; 7 plump

Ease of removal Ease of removal of kernel halves: 3 easy; 5 moderate; 7 difficult

Blanks % Percent of nuts without a kernel

Extra light % Percent of kernels in extra light category (DFA)

Light % Percent of kernels in light category (DFA)

Light amber % Percent of kernels in light amber category (DFA)

Amber % Percent of kernels in amber category (DFA)

Tip shrivel % Percent of kernels with tip shrivel like ‘Chandler’

Veins % Percent of kernels with conspicuous veins
“DFA” refers to Dried Fruit Association of California

What we claim is:

1. A new and distinct cultivar of walnut tree designated ‘Solano’ as shown and described herein.
